(19) United States
(12) Patent Application Publication
(10) Pub. No.: US 2025/0367157 A1
(43) Pub. Date: Dec. 4, 2025

(54) COMPOSITION FOR USE IN THE TREATMENT OF DISEASES ASSOCIATED WITH DYSFUNCTION OF MITOCHONDRIAE

(71) Applicant: IUF LEIBNIZ-INSTITUT FÜR UMWELTMEDIZINISCHE FORSCHUNG GMBH, Düsseldorf (DE)

(72) Inventors: Jean KRUTMANN, Düsseldorf (DE); Marc MAJORA, Düsseldorf (DE)

(21) Appl. No.: 18/877,698

(22) PCT Filed: Jun. 30, 2023
(86) PCT No.: PCT/EP2023/068042

§ 371 Date: Dec. 20, 2024

(30) Foreign Application Priority Data
Jul. 1, 2022 (EP) 22182548.2

(51) Int. Cl. A61K 31/205; A61K 31/14; A61K 31/198; A61Q 17/04

(52) U.S. Cl. CPC A61K 31/205; A61K 31/14; A61K 31/198; A61Q 17/04

(57) ABSTRACT

The present invention relates to compositions comprising a methyl group donor compound and an acetyl-CoA donor compound for use in therapy and prevention of diseases associated with dysfunction of mitochondriae, wherein the diseases associated with dysfunction of mitochondriae are nucleotide excision repair deficiency syndromes, especially Xeroderma pigmentosum A, or DNA repair deficiency syndrome ataxia telangiectasia associated with mitochondrial dysfunction. Particularly, the composition is used for therapy of nucleotide excision repair deficiency syndromes, such as Xeroderma pigmentosum A, B, C, D, E, F, G, V, the different forms of Cockayne syndrome (CS), such as Cockayne syndrome type B (CSB) and Cockayne syndrome type A (CSA), and trichothiodystrophy (TTD), or DNA repair deficiency syndrome ataxia telangiectasia associated with mitochondrial dysfunction.

The present invention relates to compositions comprising a methyl-group donor compound and an acetyl-CoA donor compound for use in therapy and prevention of diseases associated with dysfunction of mitochondriae, especially nucleotide excision repair deficiency syndromes, or DNA repair deficiency syndrome ataxia telangiectasia associated with mitochondrial dysfunction or mitochondriopathies or aging-related diseases. Particularly, the composition is used for therapy and prevention of nucleotide excision repair deficiency syndromes, such as Xeroderma pigmentosum A, B, C, D, E, F, G, V, the different forms of Cockayne syndrome (CS), such as Cockayne syndrome type B (CSB) and Cockayne syndrome type A (CSA), and trichothiodystrophy (TTD), or DNA repair deficiency syndrome ataxia telangiectasia associated with mitochondrial dysfunction, or mitochondriopathies, such as MELAS (myopathy, encephalopathy, lactic acidosis, stroke-like episodes), MERRF syndrome (myoclonus epilepsy with “ragged red fibers”), CPEO (chronic progressive external ophthalmoplegia), KSS (Kearns-Sayre syndrome), LS (Leigh syndrome) and LHON (Leber's hereditary optic neuropathy), or aging-related diseases, such as intrinsic skin aging, extrinsic skin aging and premature skin aging.

Xeroderma pigmentosum (XP) is an incurable hereditary disease that can result from certain mutations in one of eight different genes and is divided into eight different types depending on the location of said mutations. A particularly severe course of the disease is observed especially with mutations in the XPA gene. Typical of the disease is an extremely increased sensitivity of the skin to ultraviolet (UV-B) radiation. As a result, patients react to even very low levels of sun exposure with sunburn, and skin cancer can occur as early as childhood.

The risk of skin cancer can be increased by a factor of 1000 in these patients. In common parlance, those affected are referred to as “moonlight children”, as the increased sensitivity to sunlight means that they usually only spend time outdoors at night. In addition to the increased UV sensitivity and the extremely increased risk of skin cancer, the patients show many signs of an accelerated aging process. In addition to the skin, other organs such as the central nervous system are also affected. Therefore, Xeroderma pigmentosum is also counted among the so-called progeric diseases.

Investigations into the causes of Xeroderma pigmentosum have led to this disease usually being classified as a so-called DNA repair deficiency syndrome. Specifically, the above-mentioned eight genes encode proteins that play an important role in nucleotide excision repair (NER) at different sites. NER is one of the most important endogenous DNA repair systems in humans. It is the central mechanism by which UVB-induced damage to the genetic material (the nuclear DNA) of skin cells is repaired. Disruption of this repair mechanism is accordingly associated with an increased risk of skin cancer.

However, this does not explain why affected individuals also develop neuro-logical phenotypes, nor does it explain all the signs of premature aging. It can therefore be assumed that the XP proteins have further biological functions beyond their role in DNA repair.

This assumption is supported by the detection of mitochondrial and metabolic dysfunction in XPA-deficient human cells and nematodes. Fang et al. (Fang et al, Defective mitophagy in XPA via PARP1 hyperactivation and NAD+/SIRT1 reduction, Cell, 2014) suggest on the basis of functional studies that the XPA protein is important for the activation of the sirtuin Sirt1 and the elimination of damaged mitochondria through the mechanism of mitophagy.

CN 109 673 857 B discloses a composition comprising butyrate and betaine for relieving mitochondrial autophagy and relieving oxidative stress in pigs.

US 2018/256612 A1 discloses compositions comprising an exogenous ketone body and a methyl donor for treating diseases or disorders related to aging or stress, diabetes (type I or type II, obesity, neurodegenerative diseases (such as Alzheimer's disease, neurodegenerative diseases, etc.), cardiovascular diseases, muscular disorders, blood clotting disorders, inflammation, cancer, eye disorders, or mitochondrial disorders.

US 2003/078269 A1 discloses a composition comprising L-carnitine and choline for treating of insulin resistance and type II diabetes mellitus.

EP 2 792 354 A2 relates to the use of a composition comprising acetyl-L-carnitine and betaine tor treating acute and chronic hepatic encephalopathy.

US 2019/247326 A1 discloses a composition comprising vitamin B6 and butyrate for treating Wolf-Hirschhorn syndrome.

M. J. Smerdom et al. (1982), “Sodium Butyrate Stimulates DNA Repair in UV-irraditated Normal and Xeroderma Pigmentosum Human Fibroblasts”, The Journal of Biological Chemistry, 13441-13447, discloses the use of sodium butyrate in the treatment of Xeroderma Pigmentosum.

M. Scheibye-Knudsen et al. (2014), “A High Fat Diet and NAD+Rescue Premature Aging in Cockayne Syndrome”, Cell Metab., 20, 5, 840-855, discloses that high fat diet rescued the phenotype of Csb^{m/m} mice at the metabolic, transcriptomic and behavioral levels. β-hydroxybutyrate levels are increased by the high fat diet; and β-hydroxybutyrate, PARP inhibition, or NAD⁺ supplementation can activate SIRT1 and rescue CS-associated phenotypes.

Still there is the need to provide pharmaceutical compositions which can be used in the treatment and/or prevention of diseases associated with dysfunction of mitochondriae, especially nucleotide excision repair deficiency syndromes, or DNA repair deficiency syndrome ataxia telangiectasia associated with mitochondrial dysfunction or mitochondriopathies or aging-related diseases. The object of the invention is thus to provide pharmaceutical compositions for use in the therapy and prevention of diseases associated with dysfunction of mitochondriae, especially Xeroderma pigmentosum. This object may be achieved by targeting the aforementioned mitochondrial and metabolic dysfunction in XPA-deficient human cells and nematodes.

The object of the invention is surprisingly solved by a composition comprising a first compound A, which is a methyl group donor, and a second compound B, which is an acetyl-CoA donor, for use in therapy and prevention of diseases associated with dysfunction of mitochondriae, especially Xeroderma pigmentosum type A.

Especially, the object of the invention is solved by a composition comprising a first compound A, which is a methyl group donor, and a second compound B, which is an acetyl-CoA donor, for use in therapy and prevention of diseases associated with dysfunction of mitochondriae, wherein the diseases associated with dysfunction of mitochondriae are nucleotide excision repair deficiency syndromes, especially Xeroderma pigmentosum A, or DNA repair deficiency syndrome ataxia telangiectasia associated with mitochondrial dysfunction.

Surprisingly it has been found that mitochondrial dysfunction plays a key role in the pathophysiology of Xeroderma pigmentosum and that a combination of a methyl group donor compound and an acetyl-CoA donor compound is effective in at least partially compensating said mitochondrial dysfunction by increasing adenosine triphosphate (ATP) and, most likely, acetyl-CoA production in the cells. A characteristic feature of diseases like CS, XPA or TTD is a transcriptional blockade occurring most notably after induction of DNA damage by exposure with UV irradiation or other detrimental agents. It is therefore of particular interest that combined treatment with a methyl group donor and an acetyl-CoA donor can at least partially overcome the loss of mRNA transcripts seen for many genes in XPA fibroblasts with and without UV radiation. Apart from enforcing transcription or stabilization of mRNAs, the combined treatment can also increase the cellular abundance of distinct proteins either by improving protein stability or translational efficiency. It has furthermore been surprisingly found that the inventive composition may generally be used in therapy and prevention of any disease associated with dysfunction of mitochondriae.

As compound A, any tolerable methyl group donor known from the prior art may be employed. The term “methyl group donor” or “methyl donor” is well known to the person skilled in the art. The term is used in the art to describe compounds which can provide methyl groups in the human body, e.g., in metabolic pathways or DNA synthesis. By way of example, it is pointed to F. Depeint et al. (2006), “Mitochondrial function and toxicity: Role of B vitamins on the one-carbon transfer pathways”, Chemico-Biological Interactions, 163, 113-132, and to E. N. Proshkina (2020), “Key Molecular Mechanisms of Aging, Biomarkers, and Potential Interventions”, Molecular Biology, 54, 6, 777-811. Exemplary methyl group donors are trimethyl ammonium compounds such as choline and betaine, methionine, folic acid, folate, 5-methyltetrahydrofolate, vitamins B2, B6 and B12, sarcosine, serine, trimethylamine-N-oxide, and S-adenosylmethionine (SAM). In a preferred embodiment, the methyl group donor may be selected from the group consisting of compounds comprising trimethylamine-groups, sarcosine, trimethylamine-N-oxide, serine, and mixtures of two or more of them. Preferably, the methyl group donor may be selected from trimethyl ammonium compounds and mixtures of two or more of them. Preferred compounds comprising trimethylamine-groups are choline and betaine. Particularly preferred choices of methyl group donors are choline and betaine. Most preferred is choline as compound A.

In another preferred embodiment, the first compound A is selected from the group consisting of choline, betaine, sarcosine, trimethylamine-N-oxide, serine, and mixtures of two or more of them, especially selected from choline and betaine.

As compound B, any tolerable acetyl-CoA donor known from the prior art may be employed. The term “acetyl-CoA donor” is well known to the person skilled in the art. It is used in the art to describe substances or compounds from which acetyl-CoA can be generated in the human body, or substances suppressing compounds which hinder the generation of actely-CoA. By way of example, it is pointed to J. M. Frans Trijbels et al. (2004), “Chapter 5—Biochemical Diagnosis of OXPHOS Disorders” in “Oxidative Phosphorylation in Health and Disease”, Eurekah.com and Kluwer Academic/Plenum Publishers. Mixtures of two or more different acetyl-CoA donors can be employed as well. Within the meaning of the present application, acetyl-CoA donors are substances and compounds from which acetyl-CoA can be generated in the human body, or substances suppressing compounds, such as enzymes, which hinder the generation of acetyl-CoA. Exemplary acetyl-CoA donors are fatty acids, fatty acid derivatives, salts of fatty acids, salts of fatty acid derivatives, triglycerides, triglyceride derivatives, dicarboxylic acid derivatives, and pyruvate dehydrogenase kinase inhibitors.

Compounds from which acetyl-CoA can be generated are for example fatty acids as well as salts thereof. In a preferred embodiment the acetyl CoA donor is selected from fatty acids as well as salts thereof, particularly preferred are acetate or butyrate, i.e., the salts of acetic acid or butyric acid, respectively. Particularly preferred, the compound B is acetate.

Fatty acids within the meaning of the present invention are carboxylic acids with 2 to 28 carbon atoms, especially 10 to 25 carbon atoms. Their chain is aliphatic and can be saturated or unsaturated, linear, or branched. Within the meaning of the present application, both acetic acid and butyric acid are fatty acids. Any pharmaceutically acceptable salt of the above-mentioned fatty acids known from the prior art may be employed. Suitable salts are e.g., sodium salts of fatty acids, potassium salts of fatty acids, ammonium salts of fatty acids, calcium salts of fatty acids or magnesium salts of fatty acids.

A compound hindering the generation of acetyl-CoA is for example pyruvate dehydrogenase kinase (PDK). Pyruvate dehydrogenase kinase is a kinase enzyme which acts to inactivate the enzyme pyruvate dehydrogenase by phosphorylating it using ATP. There are four known isozymes of PDK in humans which all catalyze the same reaction. Accordingly, suitable compounds B are also inhibitors of pyruvate dehydrogenase kinases, for example dichloroacetate, AZD7545 ((R)-4-(3-Chloro-4-(3,3,3-trifluoro-2-hydroxy-2-methylpropan-amido)phenylsulfonyl)-N,N-dimethylbenzamide), PS10 (2-[(2,4-Dihydroxy-phenyl)sulfonyl]isoindoline-4,6-diol), dicoumarol, JX06 (Bis(morpholinothio-carbonyl)disulfide), leelamine or VER-246608 (N-[4-(2-chloro-5-methyl-4-pyrimidinyl)phenyl]-N-[[4-[[(2,2-difluoroacetyl)amino]methyl]phenyl]methyl]-2,4-dihydroxy-benzamide).

In a particularly preferred embodiment of the invention, the second compound B may be selected from fatty acids, fatty acid derivatives, salts of fatty acids, salts of fatty acid derivatives, triglycerides, triglyceride derivatives, and dicarboxylic acid derivatives, particularly from acetate, butyrate, triheptanoin, and dimethyl-α-ketoglutarate, or from pyruvate dehydrogenase kinase inhibitors. Triheptanoin is an example for a triglyceride which may be used in the present invention as compound B. Dimethyl-α-ketoglutarate is an example for a dicarboxylic acid derivative which may be used in the present invention. Preferably, the second compound B may be selected from acetate, butyrate, triheptanoin, and dimethyl-α-ketoglutarate, more preferably from acetate and butyrate. Particularly preferred, the compound B is acetate.

Fatty acid derivatives within the meaning of the present invention are modified fatty acids, such as oxylipins, hydroxy fatty acids, diols, alkenones, and wax esters. β-hydroxybutyrate is an example for a fatty acid derivative which may be used in the present invention. Salts of fatty acid derivatives within the meaning of the present invention are pharmaceutically acceptable salts of fatty acid derivatives. Suitable salts are e.g., sodium salts of fatty acid derivatives, potassium salts of fatty acid derivatives, ammonium salts of fatty acid derivatives, calcium salts of fatty acid derivatives or magnesium salts of fatty acid derivatives.

In a preferred embodiment, the inventive composition for use may consist of compound A and compound B.

A particularly preferred inventive composition for use comprises choline as compound A and an acetate as compound B, or consists thereof.

Another particularly preferred inventive composition for use comprises choline as compound A and a butyrate as compound B, or consists thereof.

Still another particularly preferred inventive composition for use comprises choline as compound A and triheptanoin as compound B, or consists thereof.

Still another particularly preferred inventive composition for use comprises choline as compound A and dimethyl-α-ketoglutarate as compound B, or consists thereof.

In a preferred embodiment, the molar ratio of the compound A to compound B is in the range of from 10:1 to 1:10. More preferably, the ratio is in the range of from 8:1 to 1:8, or 5:1 to 1:5, especially from 2:1 to 1:2, particularly 1:1.

In another preferred embodiment, the molar ratio of the compound A to compound B is in the range of from 5:1 to 10:1.

In a preferred embodiment, the concentration of compound A is in the range of from 5 to 250 mM, preferably in the range of from 15 to 150 mM, more preferably in the range of from 20 to 120 mM, and the concentration of compound B is in the range of from 1 to 100 mM, preferably in the range of from 5 to 50 mM, more preferably in the range of from 7 to 15 mM.

In another preferred embodiment, the concentration of compound A is in the range of from 25 to 100 mM and the concentration of compound B is 10 mM.

The dosage of the inventive composition and the components therein varies depending on the type of effect desired, on the weight, age, sex of the subject, and the method of administration. Generally, compositions for use can be administered in an amount based on the average weight of a subject. In a preferred embodiment, the inventive composition for use is administered in an amount of 1 to 250 mg/kg/d, preferably 2 to 240 mg/kg/d, more preferably 5 to 220 mg/kg/d, of compound A and 1 to 250 mg/kg/d, preferably 10 to 220 mg/kg/d, more preferably 50 to 200 mg/kg/d, of compound B.

In a further preferred embodiment, the inventive composition is administered in an amount of 1 to 25 mg/kg/d, preferably 2 to 20 mg/kg/d, more preferably 5 to 10 mg/kg/d, even more preferably 7 to 8 mg/kg/d, of choline as compound A and 1 to 250 mg/kg/d, preferably 10 to 220 mg/kg/d, more preferably 50 to 200 mg/kg/d, of compound B. Preferably, compound B is acetate.

In a further preferred embodiment, the inventive composition is administered in an amount of 100 to 250 mg/kg/d, preferably 120 to 240 mg/kg/d, more preferably 130 to 220 mg/kg/d, of betaine as compound A and 1 to 250 mg/kg/d, preferably 10 to 220 mg/kg/d, more preferably 50 to 200 mg/kg/d, of compound B. Preferably, compound B is acetate.

In a preferred embodiment, the inventive composition for use is administered every day.

The inventive composition for use may be administered by any suitable method known from the prior art. In a preferred embodiment, the inventive composition for use is administered orally, intravenously, or topically. The inventive composition for use may be administered topically, e.g. as compound in sunscreen products or alone. Depending on the administration, the composition for use according to the present invention may comprise any suitable additive known by the person skilled in the art, such as solvents, etc. For topical applications, antioxidants, DNA-repair-enzymes, vitamins, UV-filters, pre- or probiotic substances, etc. may be included.

In the following the rationale of the invention is further explained and it is elaborated, how mitochondrial dysfunction is linked to Xeroderma pigmentosum (XP) and how the inventive composition for use alleviates such mitochondrial dysfunction. These theoretical findings are to be understood as illustrative only and the present invention is not intended to be bound by the following theory.

### BRIEF DESCRIPTION OF THE FIGURES

FIG. 1 shows the results of the simultaneous treatment of primary XPA-deficient human fibroblasts with Choline and Acetate according to Example 1. In FIG. 1 A, the cells were either irradiated with UVB (UVB) or left unirradiated (Sham). In FIG. 1 B, the cells were either treated with Cisplatin (Cisplatin) or left untreated (Control).

FIG. 2 shows the results of the simultaneous treatment of primary XPA-deficient human fibroblasts with Choline and Acetate according to Example 2. FIG. 2 A shows the glycolytic (glycol) and mitochondrial (mito) ATP production rate. FIG. 2 B shows the results of the fluorescence microscopic analysis. FIG. 2 C shows the basal oxygen consumption rate (OCR).

FIG. 3 shows the results of the simultaneous treatment of primary XPA-deficient human fibroblasts with Choline and Acetate according to Example 3. Shown are the RNA levels of the ATP-producing glycolytic enzymes Pyruvate Kinase 1 (PKM1) [A] and Pyruvate Kinase 2 (PKM2) [B], the mitochondrial DNA-encoded ATP Synthase subunits MT-ATP6 [C] and MT-ATP8 [D], the mitochondrial antioxidant enzyme SOD2 [E], the glucose converting enzyme AKR1B1 [F], the chaperone HSPA2 [G], and the tumor suppressor gene TFPI2 [H].

FIG. 4 shows the results of the simultaneous treatment of primary XPA-deficient human fibroblasts with Choline and Acetate according to Example 4. FIG. 4 A shows the results of the analysis of cellular soluble proteins. FIG. 4 B shows the results of the analysis of DNA-associated histones.

FIG. 5 shows the results of the simultaneous treatment of primary CSB-deficient human fibroblasts with Choline and Acetate according to Example 4.

FIG. 6 shows the results of the simultaneous treatment of primary CSB-deficient human fibroblasts with Choline and Acetate according to Example 3. Shown are the RNA levels of the mitochondrial DNA-encoded genes MT-ATP6 [A], MT-ATP8 [B], MT-ND1 [C], MT-ND3 [D] and the nuclear DNA-encoded mitochondrial transcription factor gene TFAM [E].

FIG. 7 A shows the results of the simultaneous treatment of primary normal human dermal fibroblasts (NHF) with Choline (100 mM), Acetate (10 mM) and Choline (100 mM)+Acetate (10 mM) according to Example 2 (fluorescence microscopic analysis). FIG. 7 B shows the results of the simultaneous treatment of primary XPA-deficient human fibroblasts (XPA) with Choline (100 mM), Acetate (10 mM) and Choline (100 mM)+Acetate (10 mM) according to Example 2 (fluorescence microscopic analysis).

FIG. 8 shows the results of the simultaneous treatment of primary XPA-deficient human fibroblasts with Choline (100 mM), Dimethyl-α-Ketoglutarate (DMKG) (10 mM) and Choline (100 mM)+Dimethyl-α-Ketoglutarate (10 mM) according to Example 4.

FIG. 9 shows the results of the simultaneous treatment of primary XPA-deficient human fibroblasts with Choline (100 mM), Dimethyl-α-Ketoglutarate (DMKG) (10 mM) and Choline (100 mM)+Dimethyl-α-Ketoglutarate (10 mM) according to Example 3.1. Shown are the RNA levels of the mitochondrial heat shock proteins CRYAB [A], HSPA2 [B] and the inositol metabolism involved enzyme ISYNA1 [C].

FIG. 10 shows the results of the simultaneous treatment of primary XPA-deficient human fibroblasts with Choline (100 mM), Butyrate (5 mM) and Choline (100 mM)+Butyrate (5 mM) according to Example 3.1. Shown is the RNA level of the glucose converting enzyme AKR1B1.

FIG. 11 shows the results of the simultaneous treatment of primary normal human dermal fibroblasts (NHF) and primary CSB-deficient human fibroblasts (CSB) with Choline (100 mM), Acetate (10 mM) and Choline (100 mM)+Acetate (10 mM) (Chol/Ac) according to Example 2.1. FIG. 11 A shows the results of the fluorescence microscopic analysis of unirradiated (Sham) cells. FIG. 11 B shows the results of the fluorescence microscopic analysis of UVB-irradiated cells (UVB).

FIG. 12 shows the results of the simultaneous treatment of primary CSB-deficient human fibroblasts with Choline (100 mM), Acetate (10 mM) and Choline (100 mM)+Acetate (10 mM) according to Example 3.2. Shown are the RNA levels of the mitochondrial antioxidant enzyme SOD2 [A], and the mitochondrial heat shock protein HSPA2 [B].

FIG. 13 shows the results of the simultaneous treatment of primary CSB-deficient human fibroblasts with Choline (100 mM), Triheptanoin (Trihep) (100 μM) and Choline (100 mM)+Triheptanoin (100 μM) according to Example 3.2. Shown are the RNA levels of the glucose converting enzyme AKR1B1 [A], and the mitochondrial antioxidant enzyme SOD2 [B].

FIG. 14 shows the results of the simultaneous treatment of primary normal human fibroblasts from an aged donor with Choline (100 mM) and Acetate (10 mM) (Chol/Ac) according to Example 2.2.

Mitochondrial dysfunction in primary human skin fibroblasts from XPA patients (i.e. patient suffering from XP type A, where the mutations are localized in the XPA gene) has been thoroughly studied by the present inventors. It has surprisingly been observed that
(i) the XPA protein is localized in the mitochondria of human fibroblasts
(ii) the mitochondrial synthesis of the universal energy equivalent ATP, which is required for countless enzymatic processes (e.g. for DNA repair) and thus in the true sense for the survival of the cell, is massively altered in XPA cells.

In healthy cells, the generation of ATP in the mitochondria takes place via the respiratory chain and it is coupled to the consumption of oxygen. Alternatively, ATP can also be obtained via the metabolic pathway glycolysis, which, however, does not work nearly as efficiently in comparison and is therefore rather of secondary importance for the generation of ATP in healthy cells when oxygen supply is sufficient. In addition to the synthesis of ATP, the formation of the energy-rich metabolite acetyl-CoA also occurs by the enzyme pyruvate dehydrogenase (PDH) in the mitochondria. Acetyl-CoA can fulfil different functions in the cell. For example, via insertion into the citrate cycle, it can provide electrons for the mitochondrial respiratory chain, thus enabling the synthesis of ATP; alternatively, acetyl-CoA can serve as a substrate for the post-translational modification of proteins by the reversible transfer of acetyl groups (acetylation), thus modulating their function. A second pathway of post-translational modification of proteins is through the transfer of methyl groups (methylation), which is also reversible, and mitochondria are also involved in providing the metabolites required for this purpose. Acetylation and methylation have been particularly well studied using a group of nuclear DNA-binding proteins, the histones. Because of the complex dynamic pattern of these post-translational modifications at the histone level, they are also referred to as an epigenetic code that regulates all DNA-associated processes, such as transcription, replication, and repair, and thus fundamentally influences the fate of the cell. These nucleus-associated functions are thus controlled, at least indirectly, via retrograde signalling pathways through mitochondria.

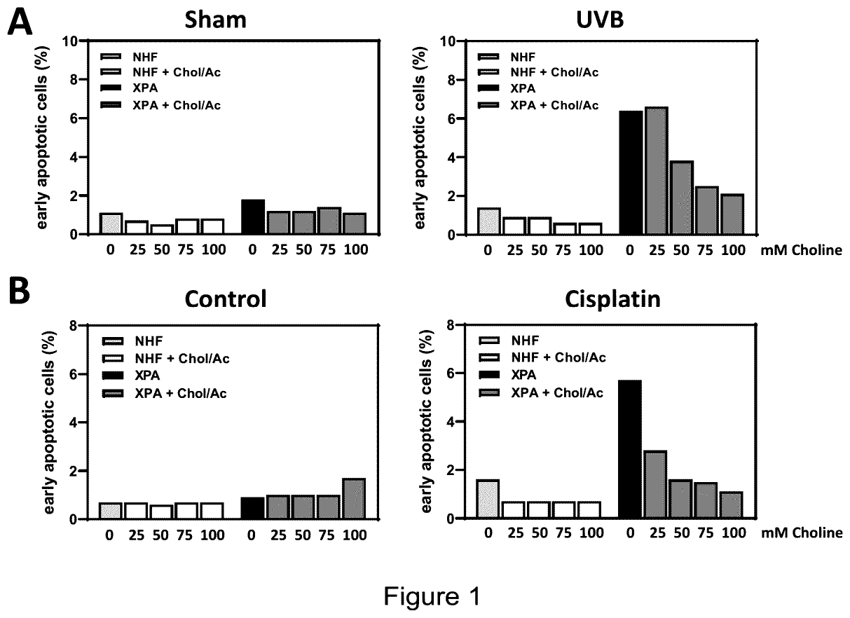

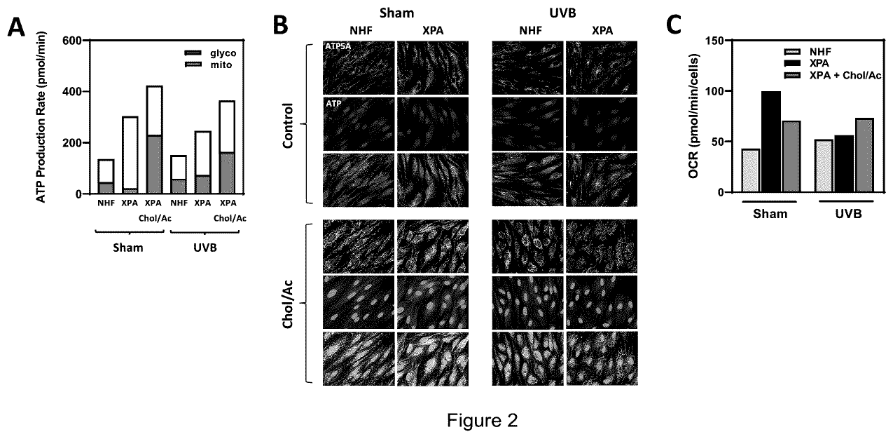

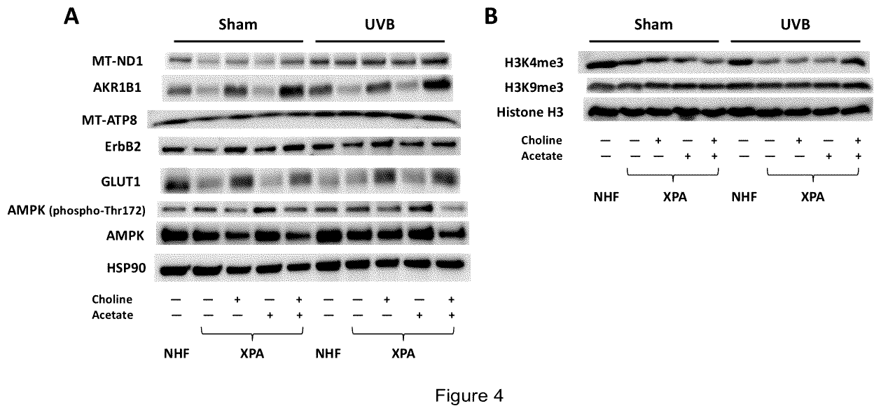

In XPA-deficient cells (e.g., skin fibroblasts from an XPA patient), the following has surprisingly been observed: Consistent with mitochondrial dysfunction, XPA-deficient fibroblasts exhibit decreased acetylation and methylation (FIG. 4 B) of histones, which is particularly evident after UVB irradiation. In addition, XPA-deficient fibroblasts exhibit increased mitochondrial oxygen consumption, suggesting increased ATP demand (FIG. 2 C). Similarly, increased phosphorylation of PDH in these cells has been detected, resulting in inactivation of the enzyme. It is therefore reasonable to assume that in XPA-deficient cells, less acetyl-CoA is available to perform acetylation of proteins such as histones and to supply the mitochondrial respiratory chain with electrons for ATP synthesis. Furthermore, the data of the present inventors show that mitochondrial ATP production is greatly reduced in XPA-deficient fibroblasts after UVB exposure (FIG. 2 A). Although the cells attempt to compensate for this loss of energy equivalents by increasing the rate of glycolysis, total ATP production is still significantly reduced compared with unirradiated XPA-deficient cells. Additionally, immunofluorescence staining with an ATP-specific antibody revealed loss of ATP in the nuclei of UVB-irradiated XPA-deficient fibroblasts (FIG. 2 B). At the same time, increased apoptosis (cell death) induction occurs in XPA-deficient fibroblasts as a result of UVB irradiation or treatment with the DNA-damaging agent Cisplatin (FIG. 1 A, XPA Sham and XPA UVB, and FIG. 1 B, XPA Control and XPA Cisplatin), accompanied by the loss of numerous proteins essential for cell function.

Based on these results, the present inventors consider that the loss of energy equivalents such as ATP and acetyl-CoA and other mitochondrial metabolites that affect methylation, for example, may be an important factor involved in the pathogenesis of XP.

The present invention suggests the following paradigm shift: Xeroderma pigmentosum proteins are not primarily or exclusively responsible for repairing DNA damage in the nucleus, but are crucial for maintaining normal function of mitochondria. Their main function is to supply the cell with energy equivalents, and this function is severely impaired in XP cells. This results in a marked deficiency of acetyl-CoA, with far-reaching consequences for a number of cellular functions. This deficiency and its consequences become most obvious when the cell is damaged, e.g. by UV radiation.

Based on these results, the present inventors treated the XPA cells with the aim of allowing the cell to increase ATP and acetyl-CoA production. Surprisingly, it could be demonstrated that this is possible in principle. Most effective in this regard was the combination of two active substances.

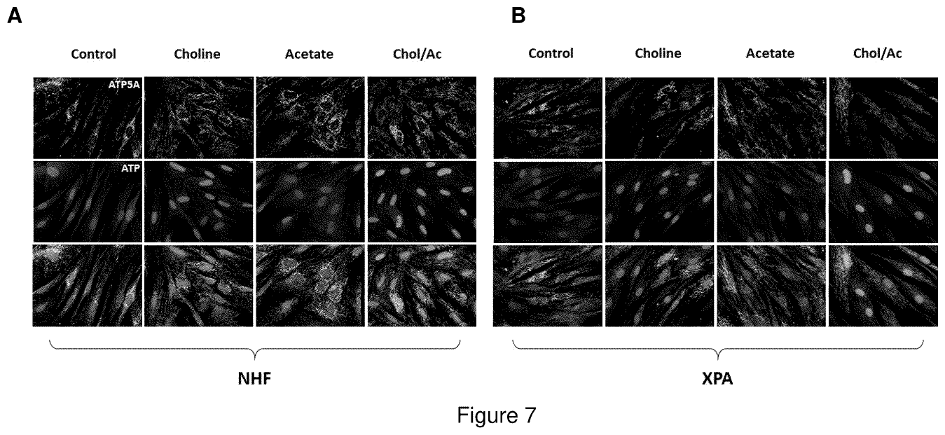

The administration of the inventive composition for use, comprising a methyl group donor and an acetyl-CoA donor, preferably a highly concentrated combination of choline and acetate, surprisingly improved the mitochondrial phenotype of XPA cells. The increased mitochondrial oxygen consumption and increased mitochondrial ATP production rates were reduced by treatment in unirradiated XPA-deficient fibroblasts. In contrast, there was an increase of mitochondrial ATP production and oxygen consumption rates in UV-irradiated XPA-deficient fibroblasts (FIGS. 2 A and C). At the same time, there was a marked increase in glycolysis-dependent ATP production, which in aggregate provided increased ATP to the cell (FIGS. 2 A and B; FIG. 7 B).

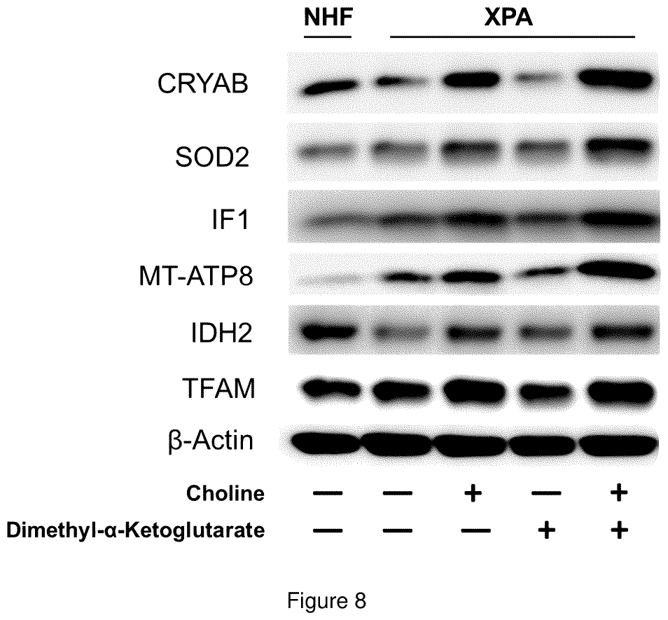

Furthermore, the inventive composition for use, comprising a methyl group donor and an acetyl-CoA donor, surprisingly reduced the increased apoptosis rate of UVB-irradiated or Cisplatin-treated XPA-deficient fibroblasts (FIGS. 1 A and B) and protected the cells from essential protein loss (FIG. 4; FIG. 8). In keeping with increased ATP production by activation of glycolysis, the anti-apoptotic effect of the combined treatment of a methyl group donor and an acetyl-CoA donor partially depends on glycolytic flux as it can be reduced by excess supply with 2-deoxy-glucose. It is further associated with increased expression of the glucose transporter GLUT1 thus ensuring sufficient fuel availability for glycolysis (FIG. 4 A).

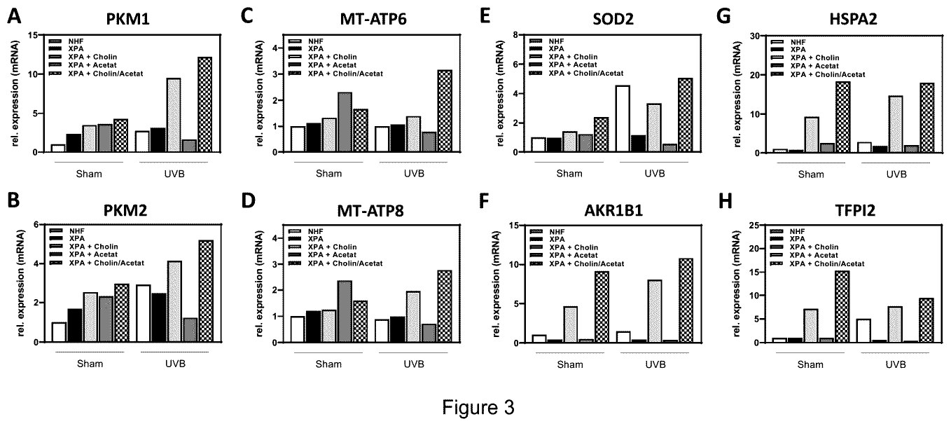

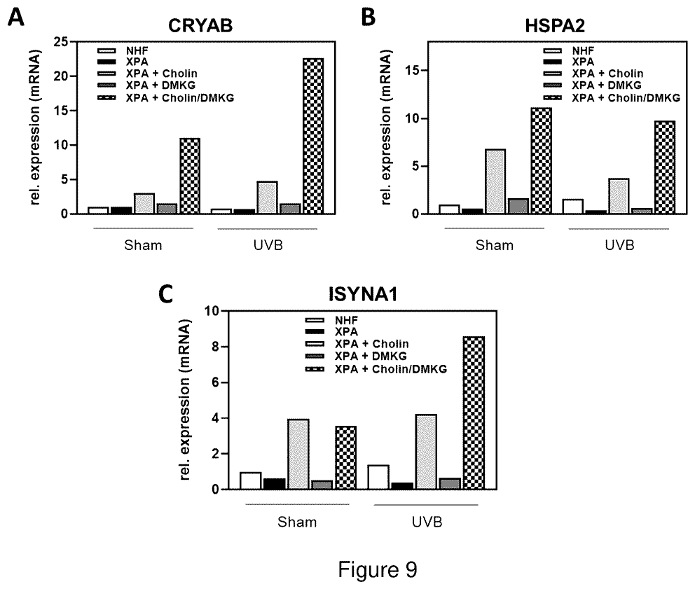

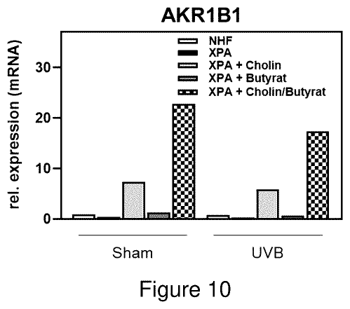

Another characteristic of XPA-deficient cells is extensive blockage of RNA new synthesis after UVB irradiation, as unrepaired DNA lesions prevent correct reading of the information. After administration of the inventive composition for use (e.g., choline/acetate; choline/DMKG; choline/butyrate), increased RNA levels of respiratory chain-associated genes in UVB-irradiated XPA-deficient fibroblasts were surprisingly observed, with the phenomenon affecting genes encoded by mitochondrial DNA as well as those encoded by nuclear DNA (FIG. 3; FIG. 9; FIG. 10). This RNA increase is also reflected by increased protein levels of mitochondrial electron transport chain-, tricarboxylic acid cycle-, and glycolysis-related proteins.

At the histone level, an increase in trimethylation could also be detected at the marker H3K4, an epigenetic modification associated with active gene expression. Trimethylation of H3K9 was also increased by the treatment (FIG. 4 B).

The totality of these effects can be observed only in the combination of the two active substances of the inventive composition for use, but not if the cells are treated only with a methyl group donor (e.g., choline) or only with an acetyl-CoA donor (e.g., acetate).

Taken together, these data show that the administration of the inventive composition for use, e.g. choline/acetate, (i) can significantly improve or normalize the mitochondrial phenotype of XPA fibroblasts, (ii) can ameliorate epigenetic alterations, and (iii) stimulates gene expression of energy metabolism-related genes or increases the stability of the respective mRNAs, thereby at least partially overcoming transcriptional blockade. Furthermore, (iv) choline/acetate treatment increases abundance of certain energy metabolism-related proteins either by increasing protein stability or by enhancing translation efficiency. Loss of ATP appears to be a critical factor driving XPA-deficient fibroblasts into apoptosis after UVB exposure and (v) administration of the inventive composition for use, e.g. choline/acetate, protects cells by stabilizing energy metabolism and inducing increased ATP production.

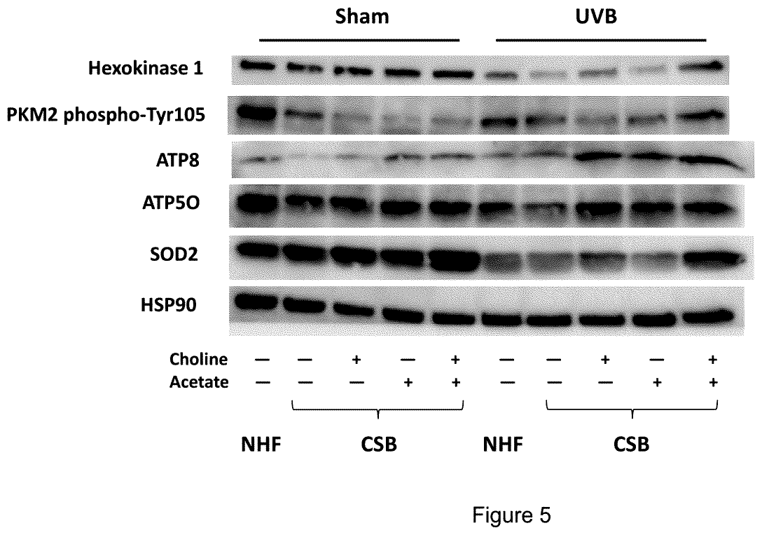

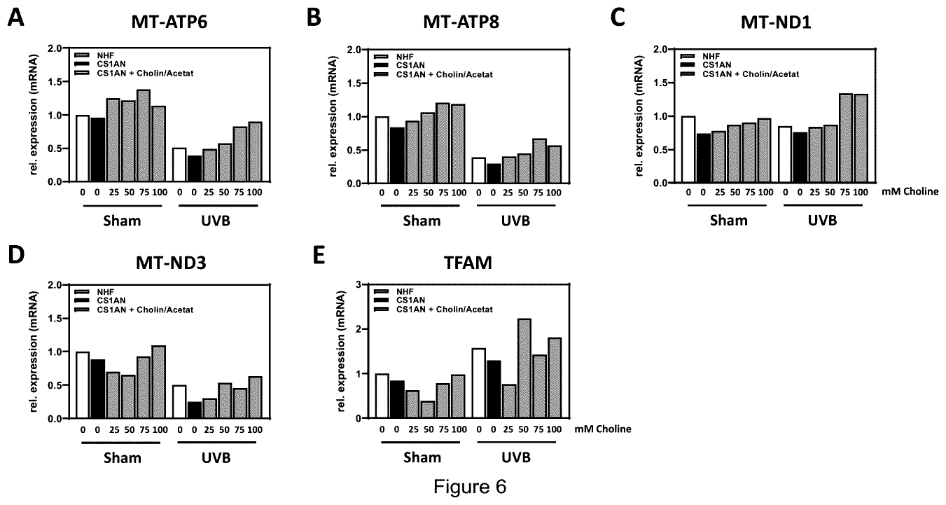

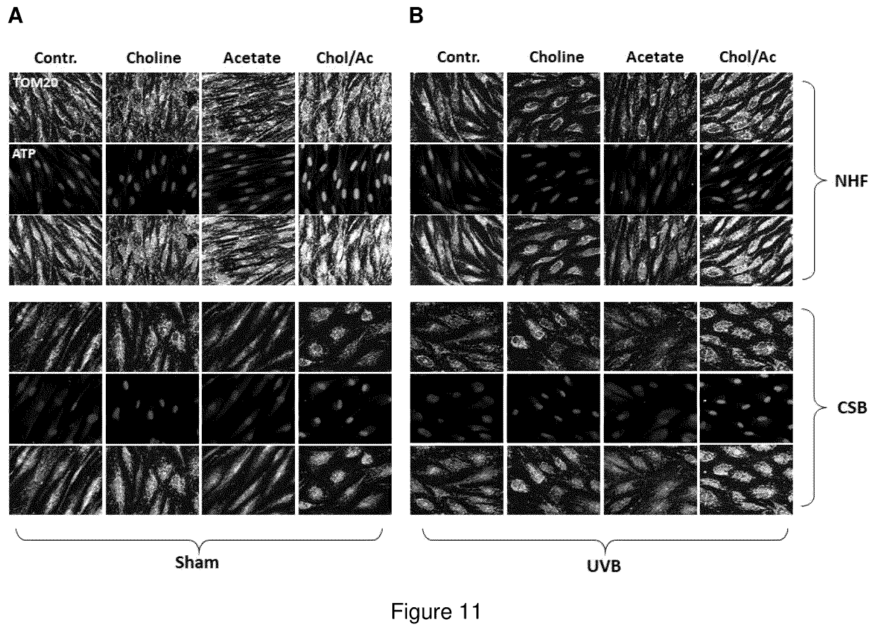

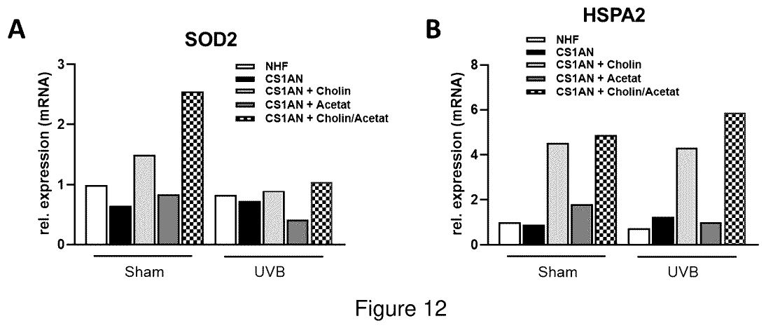

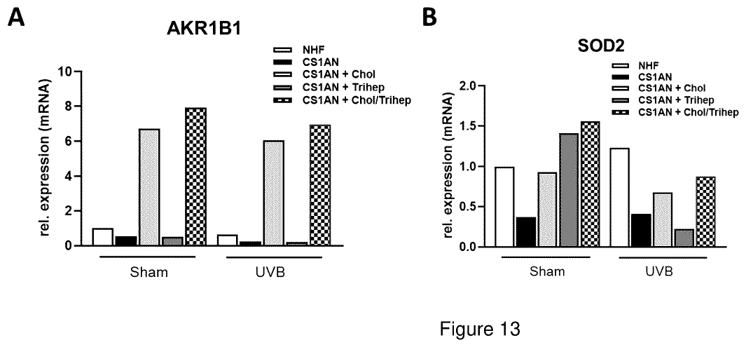

Surprisingly it has been found the therapeutic approach outlined above is also applicable to the treatment and prevention of other diseases associated with dysfunctions of the mitochondria—especially within the respiratory chain—as such diseases may also be causally linked to a deficiency of ATP and other energy-rich metabolites, similar to XPA. After administration of the inventive composition for use (e.g., choline/acetate or choline/triheptanoin), a synergistic increase of protein levels after UVB irradiation was observed in primary CSB-deficient human fibroblasts derived from Cockayne Syndrome type B patients (FIG. 5) as well as an increased expression of mitochondria-associated genes in primary CSB-deficient human fibroblasts (FIG. 6; FIG. 12; FIG. 13). Moreover, it was observed that treatment with an inventive composition (e.g., choline/acetate) increased ATP production in primary human CSB-deficient fibroblasts (FIGS. 11 A and B).

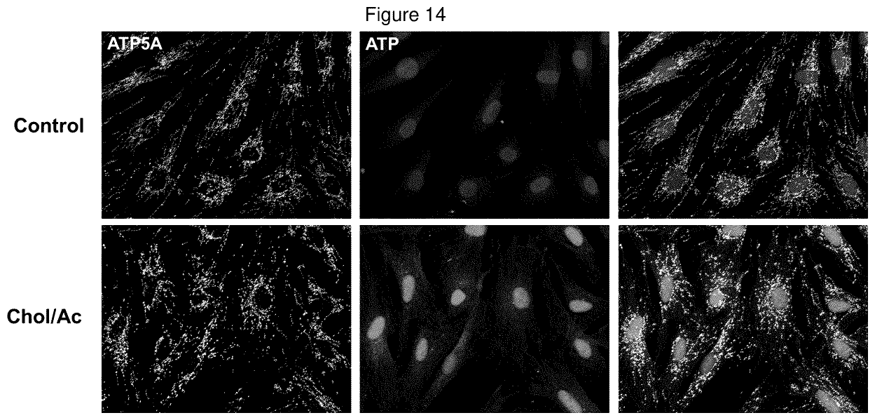

Furthermore, it was observed that treatment with an inventive composition (e.g., a combination of compound A and compound B, especially with choline and acetate) increased ATP production in primary normal human dermal fibroblasts from aged donors, in particular ATP production in the cellular nuclei (FIG. 14).

In an embodiment of the present invention, the diseases associated with dysfunction of mitochondriae are nucleotide excision repair deficiency syndromes or DNA repair deficiency syndrome ataxia telangiectasia associated with mitochondrial dysfunction or mitochondriopathies or aging-related diseases. Preferably, the nucleotide excision repair deficiency syndromes are Xeroderma pigmentosum A, B, C, D, E, F, G, V, especially Xeroderma pigmentosum A, the different forms of Cockayne syndrome (CS), such as Cockayne syndrome type B and Cockayne syndrome type A, and trichothiodystrophy (TTD). Preferably, the mitochondriopathies are MELAS (myopathy, encephalopathy, lactic acidosis, stroke-like episodes), MERRF syndrome (myoclonus epilepsy with “ragged red fibers”), CPEO (chronic progressive external ophthalmoplegia), KSS (Kearns-Sayre syndrome), LS (Leigh syndrome) and LHON (Leber's hereditary optic neuropathy). Preferably, the aging-related diseases are intrinsic skin aging, extrinsic skin aging and premature skin aging.

In a preferred embodiment, the diseases associated with dysfunction of mitochondriae are nucleotide excision repair deficiency syndromes or DNA repair deficiency syndrome ataxia telangiectasia associated with mitochondrial dysfunction.

Preferably, the nucleotide excision repair deficiency syndrome is Xeroderma pigmentosum A, B, C, D, E, F, G, V, especially Xeroderma pigmentosum A, or Cockayne syndrome type B or Cockayne syndrome type A or trichothiodystrophy (TTD).

In another preferred embodiment of the present invention, the nucleotide excision repair deficiency syndrome is Xeroderma pigmentosum A or Cockayne syndrome type B.

DNA repair deficiency syndromes are progeroid syndromes and share many features with normal aging, which are, however, more pronounced and develop faster. This is evident e.g. for skin aging. Indeed, skin fibroblasts isolated from healthy, but aged human skin show mitochondrial dysfunction. Thus, in another preferred embodiment, the diseases associated with dysfunction of mitochondriae are aging-related diseases, especially intrinsic skin aging or extrinsic skin aging or premature skin aging. Intrinsic skin aging is caused primarily by internal factors alone. It is sometimes referred to as chronological ageing and is an inherent degenerative process due to declining physiologic functions and capacities. Extrinsic skin aging is a distinctive declination process caused by external factors, which include ultra-violet radiation, cigarette smoking, air pollution, among others. Such ageing processes may include qualitative and quantitative changes and include diminished or defective synthesis of collagen and elastin in the dermis. A hallmark of skin aging is the progressive decline of mitochondrial function. In particular, markers of mitochondrial energy metabolism were found to be decreased in the skin of aged donors. Specifically, fibroblasts in aged skin typically show a decreased capacity to produce ATP. As numerous cellular processes critically involved in the maintenance of tissue homeostasis including DNA repair, transcription, replication, and translation require high amounts of ATP, loss of mitochondrial function and ATP production are thought to be involved in the manifestation of aging phenotypes in skin and other organs. Thus, generation of strategies and identification of compounds capable of increasing mitochondrial function and ATP production is considered to be of major importance for delaying or counteracting the aging process. Surprisingly it was observed that application of the inventive composition for use (e.g., choline/acetate or choline/triheptanoin) restored ATP production in aged skin fibroblasts and thus reversed their aging phenotype (FIG. 14). Particularly, the composition for use may be applied topically, e.g., as a component of sun protection products or anti-aging cosmetics, for the external treatment of aging-related diseases, such as intrinsic skin aging or extrinsic skin aging or premature skin aging.

Another preferred embodiment is the cosmetic use of the previously described composition comprising a first compound A, which is a methyl group donor, and a second compound B, which is an acetyl-CoA donor, for the treatment of intrinsic skin aging or extrinsic skin aging or premature skin aging, especially intrinsic skin aging or extrinsic skin aging. The composition may be applied topically, e.g., as a component of sun protection products or anti-aging cosmetics. The composition may comprise any suitable additive known by the person skilled in the art, such as solvents, etc. For topical applications, antioxidants, DNA-repair-enzymes, vitamins, UV-filters, pre- or probiotic substances, etc. may be included.

In another preferred embodiment, the diseases associated with dysfunction of mitochondriae are the mitochondriopathies MELAS (myopathy, encephalopathy, lactic acidosis, stroke-like episodes), MERRF syndrome (myoclonus epilepsy with “ragged red fibers”), CPEO (chronic progressive external ophthalmoplegia), KSS (Kearns-Sayre syndrome), LHON (Leber's hereditary optic neuropathy) or LS (Leigh syndrome).

The present invention thus discloses the following items:
1. Composition comprising a first compound A, which is a methyl group donor, and a second compound B, which is an acetyl-CoA donor, for use in therapy and prevention of diseases associated with dysfunction of mitochondriae, especially Xeroderma pigmentosum A.
2. Composition for use according to item 1, wherein the first compound A is selected from the group consisting of compounds comprising trimethylamine-groups, sarcosine, trimethylamine-N-oxide, serine, and mixtures of two or more of them, especially selected from choline and betaine.
3. Composition for use according to item 1 or 2, wherein the second compound B is selected from fatty acids and salts of fatty acids, particularly from acetate and butyrate, or from pyruvate dehydrogenase kinase inhibitors.
4. Composition for use according to any one of items 1 to 3, wherein the molar ratio of compound A to compound B is from 10:1 to 1:10, especially from 8:1 to 1:8, preferably from 5:1 to 1:5, especially preferred from 2:1 to 1:2, such as 1:1.
5. Composition for use according to any one of items 1 to 4, wherein the composition is administered orally, intravenously, or topically, especially topically as compound in sunscreen products or alone.
6. Composition for use according to any of items 1 to 5, wherein the concentration of compound A is in the range of from 5 to 250 mM and the concentration of compound B is in the range of from 1 to 100 mM.
7. Composition for use according to any one of items 1 to 6, wherein the diseases associated with dysfunction of mitochondriae are nucleotide excision repair deficiency syndromes or DNA repair deficiency syndrome ataxia telangiectasia associated with mitochondrial dysfunction.
8. Composition for use according to item 7, wherein the nucleotide excision repair deficiency syndrome is Xeroderma pigmentosum A, B, C, D, E, F, G, V or Cockayne syndrome type B or Cockayne syndrome type A or trichothiodystrophy (TTD).
9. Composition for use according to any one of items 1 to 6, wherein the diseases associated with dysfunction of mitochondriae are the mitochondriopathies MELAS (myopathy, encephalopathy, lactic acidosis, stroke-like episodes), MERRF syndrome (myoclonus epilepsy with “ragged red fibers”), CPEO (chronic progressive external ophthalmoplegia), KSS (Kearns-Sayre syndrome), LHON (Leber's hereditary optic neuropathy) or LS (Leigh syndrome).
10. Composition for use according to any one of items 1 to 6, wherein the diseases associated with dysfunction of mitochondriae are aging-related diseases, especially intrinsic skin aging or extrinsic skin aging or premature skin aging.

The following examples disclose the present invention in a non-limiting manner.

### EXAMPLES

### General Procedures

### Cell Culture and Treatment

Primary human fibroblasts obtained from Xeroderma pigmentosum group A (XPA) patients and Cockayne Syndrome group B (CSB) patients were purchased from Coriell Institute (USA). Normal human fibroblasts (NHF) were isolated from skin samples obtained from healthy age-matched donors undergoing skin surgery. Cells were grown in Dulbecco's Modified Eagle Medium (DMEM) supplemented with 1% glutamine, antibiotics/antimycotics (growth medium) and 15% fetal calf serum (FCS) at 37° C. in a humidified atmosphere containing 5% CO₂. For experiments, cells were detached from culture flasks by treatment with 0.05% trypsin solution (5 min/37° C.). After blocking trypsin activity by adding growth medium with 10% FCS, cells were spun down, resuspended in growth medium (15% FCS) and plated in appropriate culture dishes. Cells were allowed to grow until they reached around 80% confluency. Then medium was removed, cells were washed with phosphate-buffered saline (PBS), received growth medium containing 1% FCS and were grown for 24 hours.

Next day, cells were treated with the compounds Choline Chloride (here called Choline), or Sodium Acetate (here called Acetate), or both of them (both compounds were purchased from Sigma-Aldrich). For the treatment, appropriate amounts of the compounds were dissolved in growth medium (1% FCS) which was sterile-filtered through a 0.22 μm pore size membrane. Control growth medium (1% FCS) without added compounds was also sterile-filtered in the same way. Old growth medium was removed from the cells and was exchanged with filtered control or compound-containing growth medium. Cells were allowed to grow for additional 24 hours before they were subjected to UVB irradiation.

Irradiation was done using a self-constructed device equipped with TL 20W/12 RS SLV bulbs (Philips) emitting UVB radiation (Emission range from 290-320 nm, peak at 302 nm). The applied dose in all experiments was 20 mJ/cm². Output of the device was tested regularly with a UV meter (Waldmann) to calculate the required irradiation time. For irradiation, growth medium was removed, and cells were washed with PBS. Fresh PBS was given to the cells before they were placed in the irradiation device and were exposed to UVB for the appropriate time to reach the desired dose. Unirradiated control cells (here called Sham) were treated equally but were not exposed to UVB irradiation. After irradiation, cells were removed from the UVB device, PBS was aspirated, and all cells received fresh growth medium containing the respective compounds or control growth medium (same kind of medium they received before irradiation). Cells were incubated for 24 hours before being used for the various assays described in the subsequent examples.

### Example 1

### Apoptosis Measurement

NHF and XPA-deficient fibroblasts were grown in culture dishes in growth medium containing 15% FCS. 48 hours before UVB irradiation, medium was removed, cells were washed with PBS and received growth medium containing 1% FCS. 24 hours later, medium was removed, and cells received growth medium (1% FCS) containing varying concentrations of Choline (25, 50, 75 or 100 mM) and a constant concentration of Acetate (10 mM). Control cells received normal growth medium supplied with 1% FCS. 24 hours later, cells were either exposed to UVB or left unirradiated (Sham). After irradiation, PBS was removed, and cells received the respective growth medium with Choline/Acetate or control growth medium and were incubated for additional 24 hours. For treatment with the DNA-damaging agent Cisplatin, medium was removed, and cells were treated with medium containing Cisplatin (50 μM) (Medchem Express) dissolved in the respective growth medium either supplied with Choline/Acetate or control growth medium and were incubated for additional 24 hours. Apoptosis measurement was performed using a flow cytometry-based kit (Biolegend) according to the protocol of the manufacturer. In brief, medium was removed, cells were washed with PBS and detached from the culture dishes using trypsin by incubation for 5 min at 37° C. Trypsin was blocked by adding PBS containing 10% FCS. Each cell suspension was pooled with the respective removed medium and PBS to also collect previously detached cells. Cells were spun down and resuspended in annexin V-binding buffer containing annexin V bound to the fluorescent dye APC and propidium iodide. Cells were analyzed by using a FACSCanto flow cytometry system. The percentage of early apoptotic cells defined as annexin V (+) and propidium iodide (−) was determined.

The results of the apoptosis measurements are shown in FIG. 1. In FIG. 1 A, cells were either irradiated with UVB (UVB) or left unirradiated (Sham). In FIG. 1 B, cells were either treated with the DNA-damaging compound Cisplatin (Cisplatin) or left untreated (Control). FIG. 1 shows that the simultaneous treatment with Choline and Acetate protects primary XPA-deficient human fibroblasts from UVB- and Cisplatin-mediated apoptosis.

### Example 2

### Measurement of ATP Production Rates and Oxygen Consumption Rates

Primary NHF and XPA-deficient fibroblasts were grown in specific culture chambers designed for Seahorse XFp analyzers (Agilent) in growth medium containing 1% FCS. 24 hours before UVB irradiation, medium was removed, and cells received growth medium (1% FCS) containing Choline (100 mM) and Acetate (10 mM). Control cells received normal growth medium supplied with 1% FCS. 24 hours later, medium was removed, cells were washed with PBS and were irradiated with UVB in fresh PBS. Unirradiated control (Sham) cells grown in a second chamber were treated essentially equal but were not exposed to UVB. After irradiation, PBS was removed and cells were treated with Choline/Acetate or control medium. Mitochondrial and glycolytic ATP production rates were measured 24 hours later by using the Seahorse ATP Real-Time rate assay kit (Agilent) according to the protocol of the manufacturer.

Basal oxygen consumption rates were measured using the Seahorse Mitostress kit (Agilent) according to the protocol of the manufacturer. For normalization of results, cells were fixed with 2% paraformaldehyde after completion of the assay and stained with the nuclear fluorescence dye DAPI. Cells were counted using a fluorescence microscope (Zeiss) and results of the assays were normalized according to the counted cell numbers in each well.

The results are shown in FIG. 2 A and C. FIG. 2 A shows the results of the measurement of the glycolytic (glyco) and mitochondrial (mito) ATP production rate using Seahorse technology. FIG. 2 C shows the results of the measurement of the basal oxygen consumption rate (OCR) of NHF and XPA fibroblasts determined using Seahorse technology.

### Immunofluorescence Analysis

Primary NHF and XPA-deficient fibroblasts were grown on chamber glass slides (Sarstedt) in growth medium containing 15% FCS. 48 hours before UVB irradiation, medium was removed, cells were washed with PBS and received growth medium containing 1% FCS. 24 hours later, medium was removed, and cells received growth medium (1% FCS) containing Choline (100 mM) and Acetate (10 mM). Control cells received normal growth medium supplied with 1% FCS. 24 hours later, medium was removed, cells were washed with PBS and were irradiated with UVB in fresh PBS. Unirradiated control (Sham) cells grown in a second chamber were treated essentially equal but were not exposed to UVB. After irradiation, PBS was removed, and cells were treated with Choline/Acetate or control growth medium. 24 hours later, medium was removed, cells were washed with PBS and fixed with 2% paraformaldehyde. For immunofluorescence staining of ATP and the ATP5A subunit of the mitochondrial ATP synthase, cells were permeabilized with PBS containing 0.1% Triton X-100 and incubated with an anti-ATP antibody raised in rabbit (MyBioSource) and an anti-ATP5A antibody raised in mouse (Abcam) overnight at 4° C. Next day, antibodies were removed, and cells were washed three times with PBS containing 0.1% Triton X-100. Cells were incubated with anti-mouse and anti-rabbit secondary antibodies either labeled with a green (Alexa Fluor 488, Thermo Fisher) or with a red fluorescence dye (Alexa Fluor 594, Thermo Fisher) for 2 hours at room temperature. Antibodies were removed and cells were washed three times with PBS containing 0.1% Triton X-100. Slides were mounted using an anti-fade mounting medium (Southern Biotech) and analyzed with a fluorescence microscope (Zeiss).

The results are shown in FIG. 2 B. ATP levels were visualized by staining cells with an ATP-specific antibody (middle row) and an antibody directed against the ATP5A subunit of the mitochondrial ATP synthase (top row) and performing fluorescence microscopic analysis. The staining indicates accumulation of ATP in the cellular nuclei upon treatment with Chol/Ac.

FIG. 2 shows that the simultaneous treatment with Choline and Acetate increases ATP production in primary human XPA-deficient and normal fibroblasts.

As described herein above for FIG. 2 B, further primary normal human dermal fibroblasts (NHF) and XPA-deficient fibroblasts were grown on chamber glass slides (Sarstedt) in growth medium containing 15% FCS. Medium was removed and cells received growth medium (1% FCS) containing either Choline (100 mM), Acetate (10 mM), or a combination of Choline (100 mM) and Acetate (10 mM) (Chol/Ac). Control cells received normal growth medium supplied with 1% FCS. 24 hours later, cells received fresh medium containing the respective compounds and were incubated for another 24 hours. The cells were washed with PBS and fixed with 2% paraformaldehyde. For immunofluorescence staining of ATP and the ATP5A subunit of the mitochondrial ATP synthase, cells were permeabilized with PBS containing 0.1% Triton X-100 and incubated with an anti-ATP antibody raised in rabbit (MyBioSource) and an anti-ATP5A antibody raised in mouse (Abcam) overnight at 4° C. Next day, antibodies were removed, and cells were washed three times with PBS containing 0.1% Triton X-100. Cells were incubated with anti-mouse and anti-rabbit secondary antibodies either labeled with a green (Alexa Fluor 488, Thermo Fisher) or with a red fluorescence dye (Alexa Fluor 594, Thermo Fisher) for 2 hours at room temperature. Antibodies were removed and cells were washed three times with PBS containing 0.1% Triton X-100. Slides were mounted using an anti-fade mounting medium (Southern Biotech) and analyzed with a fluorescence microscope (Zeiss).

The results are shown in FIG. 7. ATP levels were visualized by staining cells with an ATP-specific antibody (middle row; FIGS. 7 A and B) and an antibody directed against the ATP5A subunit of the mitochondrial ATP synthase (top row; FIGS. 7 A and B) and performing fluorescence microscopic analysis. The staining indicates accumulation of ATP in the cellular nuclei upon treatment with Choline which can be further increased by adding Acetate in both NHF (FIG. 7 A) and XPA-deficient fibroblasts (FIG. 7 B). Acetate has no effect on cellular ATP abundance when given alone. The combination of Choline and Acetate (Chol/Ac) increases the cellular ATP abundance in a synergistic manner.

### Example 2.1—Fluorescence Microscopy Analysis of CSB-Deficient Fibroblasts

As described herein above for FIG. 2 B, primary NHF and CSB-deficient fibroblasts were grown on chamber glass slides (Sarstedt) in growth medium containing 15% FCS. 48 hours before UVB irradiation, medium was removed, cells were washed with PBS and received growth medium containing 1% FCS. 24 hours later, medium was removed, and cells received growth medium (1% FCS) containing either Choline (100 mM), Acetate (10 mM), or a combination of Choline (100 mM) and Acetate (10 mM) (Chol/Ac). Control cells received normal growth medium supplied with 1% FCS. 24 hours later, medium was removed, cells were washed with PBS and were irradiated with UVB (FIG. 11 B) in fresh PBS. Unirradiated control (Sham) cells (FIG. 11 A) grown in a second chamber were treated essentially equal but were not exposed to UVB. After irradiation, PBS was removed and cells were treated again with the respective compounds or control growth medium. 24 hours later, medium was removed, cells were washed with PBS and fixed with 2% paraformaldehyde. For immunofluorescence staining of ATP and the ATP5A subunit of the mitochondrial ATP synthase, cells were permeabilized with PBS containing 0.1% Triton X-100 and incubated with an anti-ATP antibody raised in rabbit (MyBioSource) and an antibody directed against the mitochondrial marker TOM20 (raised in mouse, Santa Cruz, USA) overnight at 4° C. Next day, antibodies were removed and cells were washed three times with PBS containing 0.1% Triton X-100. Cells were incubated with anti-mouse and anti-rabbit secondary antibodies either labeled with a green (Alexa Fluor 488, Thermo Fisher) or with a red fluorescence dye (Alexa Fluor 594, Thermo Fisher) for 2 hours at room temperature. Antibodies were removed and cells were washed three times with PBS containing 0.1% Triton X-100. Slides were mounted using an anti-fade mounting medium (Southern Biotech) and analyzed with a fluorescence microscope (Zeiss).

The results are shown in FIGS. 11 A and B. ATP levels were visualized by staining cells with an ATP-specific antibody (middle row in both the NHF and the CSB experiment) and an antibody directed against the mitochondrial marker TOM20 (top row in both the NHF and the CSB experiment) and performing fluorescence microscopic analysis. The staining indicates accumulation of ATP in the cellular nuclei upon treatment with Choline which can be further increased by adding Acetate in both NHF and XPA-deficient fibroblasts. The combination of Choline and Acetate increases the cellular ATP abundance in a synergistic manner.

### Example 2.2—Fluorescence Microscopic Analysis of Primary Normal Human Dermal Fibroblasts from Aged Donors

As described herein above for FIG. 2 B, primary normal human dermal fibroblasts (NHF) obtained from a healthy volunteer (age: 52 years) were grown on chamber glass slides (Sarstedt) in growth medium containing 15% FCS. Medium was removed and cells received growth medium (1% FCS) containing a combination of Choline (100 mM) and Acetate (10 mM) (Chol/Ac). Control cells (Control) received normal growth medium supplied with 1% FCS. 24 hours later, cells received fresh medium containing the respective compounds or normal medium, respectively, and were incubated for another 24 hours. The cells were washed with PBS and fixed with 2% paraformaldehyde. For immunofluorescence staining of ATP and the ATP5A subunit of the mitochondrial ATP synthase, cells were permeabilized with PBS containing 0.1% Triton X-100 and incubated with an anti-ATP antibody raised in rabbit (MyBioSource) and an anti-ATP5A antibody raised in mouse (Abcam) overnight at 4° C. Next day, antibodies were removed, and cells were washed three times with PBS containing 0.1% Triton X-100. Cells were incubated with anti-mouse and anti-rabbit secondary antibodies either labeled with a green (Alexa Fluor 488, Thermo Fisher) or with a red fluorescence dye (Alexa Fluor 594, Thermo Fisher) for 2 hours at room temperature. Antibodies were removed and cells were washed three times with PBS containing 0.1% Triton X-100. Slides were mounted using an anti-fade mounting medium (Southern Biotech) and analyzed with a fluorescence microscope (Zeiss).

The results are shown in FIG. 14. ATP levels were visualized by staining cells with an ATP-specific antibody and an antibody directed against the ATP5A subunit of the mitochondrial ATP synthase and performing fluorescence microscopic analysis. The staining indicates accumulation of ATP, in particular in the cellular nuclei, upon treatment with Chol/Ac.

### Example 3

### RNA Isolation & Real-Time PCR

Primary NHF, XPA-deficient fibroblasts (FIG. 3) or CSB-deficient fibroblasts (FIG. 6) were grown on culture dishes in growth medium containing 15% FCS. 48 hours before UVB irradiation, medium was removed, cells were washed with PBS and received growth medium containing 1% FCS. 24 hours later, medium was removed, and cells received growth medium (1% FCS) either containing Choline (100 mM), or Acetate (10 mM), or Choline (100 mM) and Acetate (10 mM) (XPA-deficient fibroblasts; FIG. 3), or various concentrations of Choline (25, 50, 75 or 100 mM) in combination with a constant concentration of Acetate (10 mM) (CSB-deficient fibroblasts; FIG. 6). Control cells received normal growth medium supplied with 1% FCS. 24 hours later, medium was removed, cells were washed with PBS and were irradiated with UVB in fresh PBS. After irradiation, PBS was removed, and cells were either treated with Choline, Acetate, Choline/Acetate or control growth medium. 24 hours later, medium was removed, cells were washed with PBS and RNA was extracted using a Direct-zol RNA kit (Zymo Research) according to the protocol of the manufacturer and stored at −80° C. 200 ng of purified RNA were used for cDNA synthesis using SuperScript reverse transcriptase (Thermo Fisher). Expression levels of the genes PKM1, PKM2, MT-ATP6, MT-ATP8, MT-ND1, MT-ND3, TFAM, SOD2, AKR1B1, HSPA2 and TFPI2 were analyzed using a CFX Connect Real-Time PCR detection system and SYBR Green Supermix (both from Bio-Rad). Expression levels of the genes mentioned above were normalized in comparison to the housekeeper genes 18S (for experiments with XPA-deficient fibroblasts) or β2-microglobulin (for experiments with CSB-deficient fibroblasts).

The results for primary XPA-deficient human fibroblasts are shown in FIG. 3. FIG. 3 shows that the simultaneous treatment with choline and acetate induces synergistic gene expression changes in primary XPA-deficient human fibroblasts. Shown are the RNA levels of the ATP-producing glycolytic enzymes Pyruvate Kinase 1 (PKM1) [A] and Pyruvate Kinase 2 (PKM2) [B], the mitochondrial DNA-encoded ATP Synthase subunits MT-ATP6 [C] and MT-ATP8 [D], the mitochondrial antioxidant enzyme SOD2 [E], the glucose converting enzyme AKR1B1 [F], the chaperone HSPA2 [G], and the tumor suppressor gene TFPI2 [H]. While Acetate alone does not increase RNA levels after UVB, it can augment Choline-induced gene expression when both compounds are given simultaneously.

The results for primary CSB-deficient human fibroblasts are shown in FIG. 6. FIG. 6 shows that the simultaneous treatment with Choline and Acetate increases expression of mitochondria-associated genes in primary CSB-deficient human fibroblasts (CS1AN). Shown are the RNA levels of the mitochondrial DNA-encoded genes MT-ATP6 [A], MT-ATP8 [B], MT-ND1 [C], MT-ND3 [D] and the nuclear DNA-encoded mitochondrial transcription factor gene TFAM [E].

### Example 3.1—Gene Expression Changes in Primary XPA-Deficient Human Fibroblasts

As described herein above for FIG. 3, Primary NHF and XPA-deficient fibroblasts were grown on culture dishes in growth medium containing 15% FCS. 48 hours before UVB irradiation, medium was removed, cells were washed with PBS and received growth medium containing 1% FCS. 24 hours later, medium was removed, and cells received growth medium (1% FCS) either containing Choline (100 mM), Dimethyl-α-Ketoglutarate (DMKG) (10 mM), or a combination of Choline (100 mM) and DMKG (10 mM) (FIG. 9), or Choline (100 mM), Butyrate (5 mM), or a combination of Choline (100 mM) and Butyrate (5 mM) (FIG. 10). Control cells received normal growth medium supplied with 1% FCS. 24 hours later, medium was removed, cells were washed with PBS and were irradiated with UVB in fresh PBS. After irradiation, PBS was removed and cells were again either treated with Choline, DMKG, Choline/DMKG, or with Choline, Butyrate or Choline/Butyrate, or with control growth medium. 24 hours later, medium was removed, cells were washed with PBS and RNA was extracted using a Direct-zol RNA kit (Zymo Research) according to the protocol of the manufacturer and stored at −80° C. 200 ng of purified RNA were used for cDNA synthesis using SuperScript reverse transcriptase (Thermo Fisher). Expression levels of selected genes were analyzed using a CFX Connect Real-Time PCR detection system and SYBR Green Supermix (both from Bio-Rad). Expression levels of the selected genes were normalized in comparison to the housekeeper gene 18S.

The results for the experiments with Choline and Dimethyl-α-Ketoglutarate (DMKG) are shown in FIG. 9. Expression of selected genes was analyzed 24 hours after UVB irradiation. Shown are the RNA levels of the mitochondrial heat shock proteins CRYAB [A], HSPA2 [B] and the inositol metabolism involved enzyme ISYNA1 [C]. While DMKG alone has only little (or even no) effect on most RNA levels, it can augment Choline-induced gene expression of CRYAB, HSPA2 and ISYNA1 synergistically when both compounds are given simultaneously.

The results for the experiments with Choline and Butyrate are shown in FIG. 10. Expression of selected genes was analyzed 24 hours after UVB irradiation. Shown are the RNA levels of the glucose converting enzyme AKR1B1. While Butyrate alone has only little effect on the RNA levels of AKR1B1, it can augment expression of the gene synergistically when given simultaneously with Choline.

### Example 3.2—Gene Expression Changes in Primary CSB-Deficient Human Fibroblasts

As described herein above for FIG. 6, primary NHF or CSB-deficient fibroblasts were grown on culture dishes in growth medium containing 15% FCS. 48 hours before UVB irradiation, medium was removed, cells were washed with PBS and received growth medium containing 1% FCS. 24 hours later, medium was removed and cells received growth medium (1% FCS) either containing Choline (100 mM), Acetate (10 mM), or a combination of Choline (100 mM) and Acetate (10 mM) (FIG. 12), or Choline (Chol) (100 mM), Triheptanoin (Trihep) (100 μM), or a combination of Choline (100 mM) and Trihep (100 μM) (Chol/Trihep) (FIG. 13). Control cells received normal growth medium supplied with 1% FCS. 24 hours later, medium was removed, cells were washed with PBS and were irradiated with UVB in fresh PBS. After irradiation, PBS was removed and cells were either treated with Choline, Acetate or Choline/Acetate, or with Choline, Triheptanoin or Choline/Triheptanoin, or with control growth medium.

In the case of the treatment with Choline and Acetate, the expression of selected genes was analyzed 48 hours after UVB irradiation. In the case or the treatment with Choline and Triheptanoin, the expression of selected genes was analyzed 24 hours after UVB irradiation. In both cases, medium was removed, cells were washed with PBS and RNA was extracted using a Direct-zol RNA kit (Zymo Research) according to the protocol of the manufacturer and stored at −80° C. 200 ng of purified RNA were used for cDNA synthesis using SuperScript reverse transcriptase (Thermo Fisher). Expression levels of selected genes were analyzed using a CFX Connect Real-Time PCR detection system and SYBR Green Supermix (both from Bio-Rad). Expression levels of the selected genes were normalized in comparison to the housekeeper gene 18S β2-microglobulin.

The results for the experiments with Choline and Acetate are shown in FIG. 12. Expression of selected genes was analyzed 48 hours after UVB irradiation. Shown are the RNA levels of the mitochondrial antioxidant enzyme SOD2 [A], and the mitochondrial heat shock protein HSPA2 [B]. While Acetate alone has only little or even no effect on the RNA levels of the depicted genes, it can augment their expression synergistically when given simultaneously with Choline.

The results for the experiments with Choline (Chol) and Triheptanoin (Trihep) are shown in FIG. 13. Expression of selected genes was analyzed 24 hours after UVB irradiation. Shown are the RNA levels of the glucose converting enzyme AKR1B1 [A], and the mitochondrial antioxidant enzyme SOD2 [B]. While Triheptanoin alone has only little or even no positive effect on the RNA level of AKR1B1, and SOD2 (after UVB irradiation), it can augment expression of these genes synergistically when given simultaneously with Choline.

### Example 4

### Protein Extraction & Western Blot Analysis

Primary NHF, XPA-deficient fibroblasts (FIG. 4) and CSB-deficient fibroblasts (FIG. 5) were grown on culture dishes in growth medium containing 15% FCS. 48 hours before UVB irradiation, medium was removed, cells were washed with phosphate buffered saline (PBS) and received growth medium containing 1% FCS. 24 hours later, medium was removed, and cells received growth medium (1% FCS) either containing Choline (100 mM), or Acetate (10 mM), or Choline (100 mM) and Acetate (10 mM). Control cells received normal growth medium supplied with 1% FCS. 24 hours later, medium was removed, cells were washed with PBS and were irradiated with UVB in fresh PBS. After irradiation, PBS was removed, and cells were either treated with Choline, Acetate, Choline/Acetate or control growth medium. 24 hours later, medium was removed, cells were washed with ice cold PBS and covered with fresh ice cold PBS. Using a cell lifter, cells were detached from the culture dishes, transferred to an Eppendorf tube, spun down and resuspended in ice cold RIPA lysis and protein extraction buffer supplied with proteinase and phosphatase inhibitors. Samples were kept on ice for 20 minutes before being spun down at 14000 rpm for 15 minutes at 4° C. Supernatants with solubilized proteins were collected, snap frozen in liquid nitrogen and stored at −80° C. Pellets containing RIPA-insoluble proteins including histones were also stored at −80° C. Extraction of histones was performed using the EpiQuik Total Histone Extraction kit (Epigentek). Pellets were dissolved in ice cold lysis buffer and placed on ice for 30 minutes. Samples were spun down and supernatants were collected and neutralized with Balance buffer supplied with DTT. Histone samples were stored at −80° C. For Western Blot detection, protein aliquots were separated by SDS-polyacrylamide gelelectrophoresis before being transferred on PVDF membranes by using the Trans-Blot Turbo Transfer system (Bio-Rad). Membrane slices were incubated with the indicated antibodies overnight on a shaker at 4° C. After washing, membrane slices were incubated with appropriate secondary antibodies coupled to horseradish peroxidase for 2 hours at room temperature. After washing, membrane slices were incubated with ECL western blotting substrate and protein bands were detected with an Odyssey XF Imaging system (LI-COR).

The results for primary XPA-deficient human fibroblasts are shown in FIG. 4. FIG. 4 A shows the results of the Western Blot analysis of cellular soluble proteins. FIG. 4 B shows the results of the Western Blot analysis of DNA-associated histones. FIG. 4 shows that the simultaneous treatment with Choline and Acetate induces synergistic changes in protein abundance, cellular signaling and epigenetic markers in primary XPA-deficient human fibroblasts. MT-ND1 and AKR1B1 show a synergistic accumulation with and without UVB irradiation, while MT-ATP8 particularly accumulates after UVB irradiation and ErbB2 particularly without UVB irradiation. GLUT1 levels are equally increased by single Choline or Choline+Acetate treatment. AMPK, which functions as an energy sensor stabilized and phosphorylated by cellular energy demand, displays a synergistic downregulation of total protein level and a decrease of phosphorylation in particular after UVB indicating high abundance of energy equivalents by Choline+Acetate treatment.

The results for primary CSB-deficient human fibroblasts are shown in FIG. 5. FIG. 5 shows that the simultaneous treatment with Choline and Acetate induces synergistic changes in protein abundance in primary CSB-deficient human fibroblasts. Synergistic increase of protein levels by Choline+Acetate treatment is particularly visible after UVB irradiation.

As described herein above for FIG. 4, primary NHF and XPA-deficient fibroblasts were grown on culture dishes in growth medium containing 15% FCS. After reaching confluency, XPA-deficient fibroblasts were treated with growth medium (1% FCS) either containing Choline (100 mM), Dimethyl-α-Ketoglutarate (10 mM), or a combination of Choline (100 mM) and Dimethyl-α-Ketoglutarate (10 mM). Control cells received normal growth medium supplied with 1% FCS. 24 hours later, medium was removed and cells were either treated with Choline, Dimethyl-α-Ketoglutarate, Choline/Dimethyl-α-Ketoglutarate, or control growth medium. After 24 hours of incubation, medium was removed, cells were washed with ice cold PBS and covered with fresh ice cold PBS. Using a cell lifter, cells were detached from the culture dishes, transferred to an Eppendorf tube, spun down and resuspended in ice cold RIPA lysis and protein extraction buffer supplied with proteinase and phosphatase inhibitors. Samples were kept on ice for 20 minutes before being spun down at 14000 rpm for 15 minutes at 4° C. Supernatants with solubilized proteins were collected, snap frozen in liquid nitrogen and stored at −80° C. For Western Blot detection, protein aliquots were separated by SDS-polyacrylamide gelelectrophoresis before being transferred on PVDF membranes by using the Trans-Blot Turbo Transfer system (Bio-Rad). Membrane slices were incubated with the indicated antibodies overnight on a shaker at 4° C. After washing, membrane slices were incubated with appropriate secondary antibodies coupled to horseradish peroxidase for 2 hours at room temperature. After washing, membrane slices were incubated with ECL western blotting substrate and protein bands were detected with an Odyssey XF Imaging system (LI-COR).

The results are shown in FIG. 8. The mitochondrial proteins CRYAB, SOD2, IF1, MT-ATP8, IDH2—and TFAM to a lesser extent—show a synergistic accumulation upon treatment with both compounds, Choline and Dimethyl-α-Ketoglutarate.

1. A method for the treatment of a disease associated with dysfunction of mitochondriae, comprising administering i) a first compound A, which is a methyl group donor, and
ii) a second compound B, which is an acetyl-CoA donor.

2. The method of claim 1, wherein the first compound A is selected from the group consisting of a trimethylamine, sarcosine, a trimethylamine-N-oxide, serine, and mixtures of two or more of them.

3. The method of claim 1, wherein the second compound B is selected from fatty acids, fatty acid derivatives, salts of fatty acids, salts of fatty acid derivatives, triglycerides, triglyceride derivatives, and dicarboxylic acid derivatives.

4. The method of claim 1, wherein the molar ratio of compound A to compound B is from 10:1 to 1:10.

5. The method of claim 1, wherein the administration is orally, intravenously, or topically.

6. The method of claim 1, wherein the concentration of compound A is in the range of from 5 to 250 mM and the concentration of compound B is in the range of from 1 to 100 mM.

7. The method of claim 1, wherein the disease associated with dysfunction of mitochondriae is selected from one or more of:
i) nucleotide excision repair deficiency syndromes, and
ii) DNA repair deficiency syndrome ataxia telangiectasia.

8. The method of claim 7, wherein the nucleotide excision repair deficiency syndrome is:
i) Xeroderma pigmentosum A,
ii) Xeroderma pigmentosum B,
iii) Xeroderma pigmentosum C,
iv) Xeroderma pigmentosum D,
v) Xeroderma pigmentosum E,
vi) Xeroderma pigmentosum F,
vii) Xeroderma pigmentosum G,
viii) Xeroderma pigmentosum V,
ix) Cockayne syndrome type A,
x) Cockayne syndrome type B, or
xi) trichothiodystrophy (TTD).

9. The method of claim 2, wherein compound A is a trimethyl ammonium compounds or a mixture of two or more trimethyl ammonium compounds.

10. The method of claim 2, wherein compound A is one or more of choline and betaine.

11. The method of claim 3, wherein compound B is selected from the group consisting of acetate, butyrate, triheptanoin, dimethyl-α-ketoglutarate, and pyruvate dehydrogenase kinase inhibitors.

12. The method of claim 4, wherein the ratio is selected from:
i) 8:1 to 1:8,
ii) 5:1 to 1:5,
iii) 2:1 to 1:2, or
iv) 1:1.

13. The method of claim 5, wherein the administration is topical.

14. The method of claim 13, wherein the administration further comprises administering a sunscreen product.

15. A pharmaceutical composition comprising:
a first compound A, which is a methyl group donor, and
a second compound B, which is an acetyl-CoA donor.

16. The pharmaceutical composition of claim 15, wherein compound A is selected from the group consisting of one or more of a trimethylamine, sarcosine, trimethylamine-N-oxide, serine, and mixtures thereof.

17. The pharmaceutical composition of claim 15, wherein compound B is selected from the group consisting of fatty acids, fatty acid derivatives, salts of fatty acids, salts of fatty acid derivatives, triglycerides, triglyceride derivatives, dicarboxylic acid derivatives, and mixtures thereof.

18. The pharmaceutical composition of claim 15, wherein the molar ratio of compound A to compound B is from:
i) 10:1 to 1:10,
ii) 8:1 to 1:8,
iii) 5:1 to 1:5,
iv) 2:1 to 1:2, or
v) 1:1.

19. The pharmaceutical composition of claim 15, wherein the composition is formulated for oral, intravenous, or topical administration.

20. The pharmaceutical composition of claim 15, wherein the concentration of compound A is in the range of from 5 to 250 mM and the concentration of compound B is in the range of from 1 to 100 mM.
